(19) United States
(12) Patent Application Publication
(10) Pub. No.: US 2005/0158247 A1
(43) Pub. Date: Jul. 21, 2005

(54) Nasal peptide pharmaceutical formulation

(75) Inventors: Paolo Alberto Veronesi, Milano (IT); Pablo Rodriguez, Cordoba (AR)

Correspondence Address: BURNS DOANE SWECKER & MATHIS L L P, POST OFFICE BOX 1404, ALEXANDRIA, VA, 22313-1404, US

(73) Assignee: Therapicon SRL, Milano (IT)

(21) Appl. No.: 10/516,613

(22) PCT Filed: Jun. 24, 2003
(86) PCT No.: PCT/EP03/06641

(30) Foreign Application Priority Data
Jul. 29, 2002 (IT) MI2002A 001684

(51) Int. Cl. A61L 9/04; A61K 38/22

(52) U.S. Cl. 424/45; 514/2; 514/16

(57) ABSTRACT

The invention provides a pharmaceutical formulation comprising:

(1) THAM, which is tris(hydroxymethyl)aminomethane, as a selective absorbefacient to enhance through the nasal mucosa-lined epithelium the absorption of substances of peptide nature; and

(2) a therapeutically effective amount of active nasal peptide, its pharmaceutically acceptable salt or its peptidic fragment; in a pharmaceutically acceptable, aqueous liquid diluent or carrier, said formulation being in a form suitable for nasal administration.

This invention relates to a pharmaceutical formulation for nasal administration comprising a combination of a pharmaceutically active peptide, from natural, synthetic or recombinant origin, a peptide hormone, a polypeptide or any of their pharmaceutically acceptable salts or any of their peptidic fragments, a personalized peptide or a mixture thereof (hereinafter, for the sake of convenience, defined “nasal peptide”) as a therapeutically active ingredient, and of the absorbefacient and stabilizer THAM, namely tris (hydroxymethyl) aminomethane, in a pharmaceutically acceptable liquid diluent or carrier, and particularly is concerned with a ready-to-use or reconstituted aqueous pharmaceutical solution for nasal administration. In fact the selected absorbefacient THAM is the only hydrogen-ion acceptor amine, without any marked toxicity (most amines produce marked toxic effects in vivo when used in dose sufficient quantities), which can physiologically and reversibly depolarize the nasal mucosa epithelial cells, thus exerting absorbefacient activity by enhancing the permeability and improving the efficiency of active absorption through the nasal mucosa. Therefore THAM is the unique and significant aminic absorbefacient agent characterizing the pharmaceutical formulation of the present invention, which may be therefore dosed through nasal mucosa. THAM represents also a remarkable stabilizer for the nasal peptide of the pharmaceutical formulation of the invention. The present invention also relates to a method for producing the pharmaceutical formulation either as a ready-to-use or as a reconstituted solution, which can be put up in mono-disposable or in multidose delivery system device.

### BACKGROUND ART

Pharmacologically active peptides, such as buserelin, insulin, desmopressin and many other medium and long-chain polypeptides of varying, well documented pharmaceutical utility and used at present as therapeutic drugs, are easily degraded with enzymes in the human stomach and intestine and are easily metabolized in the human lever. Therefore such polypeptides are difficult to have them absorbed through gastrointestinal tracts and to elicit their intrinsic pharmaceutical effects in a patient's body. Thus, hereto, peptides have been generally administered as various injections such as hypodermic, intramuscular and intravenous injections.

However, the patient experiences pain and irritation such as, for example, injury and tissue necrosis during long-term peptide administration of the injections and there is a potential risk for infections caused from communicable diseases.

Also the most recent formulations for long-term injectable administration of some peptides (U.S. Pat. No. 5,582,591; U.S. Pat. No. 5,776,885 and U.S. Pat. No. 6,376,461) have not satisfactorily solved the patient's compliance. The liberation rate of the peptide presents a high peak during the initial period (the release of the peptide is not as gradual as desired), the quantity of residual chlorate organic solvents, used for these classical processes, is remarkable and the final sterilization process of the formulated product is performed only by gamma-radiation, with the consequent possible risks. The above are some of the negative profiles still representing the major unsolved technical problems of the long-lasting parenteral administration route of peptides.

Other alternative administration methods for peptides have been also tentatively proposed in the past, such, for example, rectal administration as a suppository (J. Pharm. Pharmacol., 33, 334, 1981), endotracheal administration (Diabetes, 20, 552, 1971) and eyedropping administration (J. of Diabetic Society, Summary, 237, 1974). However, none of these attempts have yet been put to practical use because of unsatisfactory absorption rate, great variation in absorption, irritation caused from the absorption enhancers and from preserving or auxiliary ingredients.

For all these reasons, extensive studies have been made during the most recent years to administer peptide compounds through mucosae such as nasal mucosa. Therefore peptide containing pharmaceutical formulations for administration through nasal mucosa are highly desired and their development has attracted the interest of many researchers during the past decade.

Nevertheless, due to the fact that peptide compounds are remarkably unstable in aqueous solutions and, when not suitably formulated, may easily degrade, lose their activity and develop undesired degradation products, many authors have concentrated their efforts to develop powder compositions for nasal administration (e.g. EP 0 302 772; EP 0 468 182 and WO 99/59543).

The attempts to obtain pharmaceutical aqueous solutions were at present rather limited and often applicable solely to a specific peptide, as for example the nasal administration of insulin (EP 94157), vasopressin (EP 55066-517; JP 55055-120) and peptides of different formula (DE 2.256.445; DE 2.758.463; BE 860.717; SA 68/421). Most of these publications teach only a specific technical solution for one specific peptide, so that there is still the urgent need to have a general pharmaceutical formulation suitable and equivalent not only to those compositions of peptides already formulated for nasal administration, but also conveniently applicable to a larger number of peptides which are used at present only by parenteral route.

However, additional technical problems can arise: sometimes the liquid drug readily runs out after administration; in other cases the absorption enhancer and/or preserving agent produces undesirable adverse effects, as for example the use of benzalkonium chloride (Amer. J. Ophtalmol. 105, 6, 1988, p 670-73; Contact Dermatitis 17, 1, 1987, p 41-2; Cutis, 39, 5, 1987, p 381-83) or of chlorobutanol (Acta Otolaryng. 70, 1970, p 16-26; Merck Index Twelfth Edition entry n. 2148; U.S. Pat. 5,759,565), while there are further problems that safety and stability of peptide are affected due to the addition of surface agents, other auxiliary ingredients and accidental incorporation of microorganisms.

Moreover, in most adults the capacity of the human nasal cavity surface for holding aqueous solutions of most nasally administered agents is less than 400 microliters, while, on the other hand, about 100 microliters is the lowest dosing volume that can be conveniently reproduced by single actuations of the metered device. However, for an efficient systemic absorption of nasally administered therapeutics, the vehicle carrying the drug must remain in contact with the mucus-lined epithelium for sufficient period of time.

Viscosity modifying agents like methylcellulose or crospovidone or povidone have sometimes been used in an attempt to prolong the contact of the preparation and of the nasal epithelium (EP 0122036). Nevertheless, a recent publication of experimental studies in rabbits clearly show that the increased viscosity influence negatively the peptide intranasal absorption. Similarly, the same study (Int. Jour. of Pharm., 147, 1997, p 233-242) also demonstrates the negative influence of tonicity on the intranasal absorption of a peptide. Isotonic solutions are therefore to be avoided to optimize the intranasal absorption of a peptide.

Despite the fact that nasal administration of active peptides is described in the literature, different authors have reported either limited achievements and partially satisfactory results or contradictory findings : Experientia, 1969 Nov. 15, 25 (11), p 1195-6; Lancet, 1974 May 4, 1 (7862), p 865; Antimicrobial Agents Chemother., 1978 Oct., 14 (4), p 596-600; Lancet, 1979 Aug. 4, 2 (8136), p 215-7; Br. Med. J., 1982, 284 (6312), p 303-6; Aerosols in Medicine, Elsevier Scientific, 1983, p 346.

Another technical aspect practically ignored in literature, but very important and well known to a skilled person in the art, is the stability of peptides, especially when formulated. In fact the most preferred storage conditions for peptides are: the physical solid state, temperatures around 0° C. and accurate protection from oxygen, which is mainly responsible of the degradation processes involving the oxidation of the disulphide bonds and/or aminic radicals, which characterize the three-dimensional structure of the peptides and their biological activity, so that it is very important to avoid or limit as much as possible such accidental contact.

Similarly, equivalent conditions shall be conveniently observed when the peptides are prepared as pharmaceutical formulations. However it has been observed that isotonicity and the pH range of the solutions seem not to be essential conditions neither for enhancing the absorption of the peptides nor for their galenical compositions, while the use of nitrogen is a preventive measure well known since many decades.

Another striking, desired effect is also to stabilize (protect from oxygen) the pharmaceutical compositions comprising the therapeutic peptide. Moreover, the stability requirement for such a pharmaceutical formulation shall not be limited only to the shelf life before its use, but it shall be aimed also to its in-use stability after opening, particularly when a multidose container is used, as it is recommended by recent note for guidance adopted by some regulatory authorities, as for example The European Agency for the Evaluation of Medicinal Products (EMEA)-Note for Guidance CPMP/QWP/2934/99, September 2001, available at the Internet site hppt:/www.emea.eu.int/.

### OBJECTS OF THE INVENTION

Thus, the problem underlying the present invention is to create the novel and general pharmaceutical formulation for administration through the nasal mucosa, comprising the convenient combination of any selected pharmaceutically active nasal peptide and of the absorbefacient and stabilizer THAM, in order to achieve constant absorption rates, optimal therapeutic dose levels of the nasal peptide and to improve the patient's compliance. A further scope underlying the present invention is to preserve the nasal peptide from safety and stability problems (oxidation of the disulphide bridges) of the pharmaceutical formulation, by improving not only the shelf life before opening, but also the in-use stability, when the multidose container is opened. A further target underlying the present invention is a method for producing the pharmaceutical formulation of the invention as ready-to-use or as reconstituted solution, which may be conveniently put up in a mono-disposable or in a multidose dispensing system device.

### DESCRIPTION OF THE INVENTION

Surprisingly this has been attained by the present invention.

This invention is based on the unexpected recognition that pharmaceutical formulations for nasal administration comprising a combination of a pharmaceutically active nasal peptide and of the absorbefacient and stabilizer THAM in a pharmaceutically acceptable liquid diluent or carrier, said aqueous solution comprising optionally other pharmaceutically acceptable auxiliary additives, significantly fulfil the above requirements and satisfactorily overcome most of the reported technical problems of these formulations.

Moreover viscosity modifying agents like methylcellulose or crospovidone or povidone have been purposely avoided in the composition because of its adverse effect on the intranasal absorption of a peptide and also for their possible negative effect on the long term stability of the composition, with formation of opalescent micelles or precipitating agglomerates. Similarly the isotonicity has been purposely avoided, since isotonic solutions seem to negatively influence the intranasal absorption of a peptide.

It has been found unexpectedly that such pharmaceutical formulation is remarkably suitable for nasal administration. When applied to the mucus-lined epithelium as ready-to-use or as reconstituted solution, exhibits the desired pharmacological effect, as much as necessary to elicit the desired therapeutic activity in relation to the administration through other routes, and presents remarkable stabilizing properties, thus reducing the risk of development of degradation and/or of inactivation of the nasal peptide during the shelf life period before its use, but particularly improving the in-use stability after opening, when put up in a multidose delivery system device, which is, as it is often the case, subsequently stored for months before use.

Thus in a first aspect, according to the present invention, the pharmaceutical formulation for nasal administration comprises:
(1) a therapeutically effective amount of a pharmaceutically active nasal peptide or its salt or fragment, as therapeutically active ingredient; and
(2) the absorbefacient and stabilizer THAM; in a pharmaceutically acceptable liquid diluent or carrier suitable for application to the mucus-lined epithelium of the nasal mucosa, said aqueous solution comprising optionally other pharmaceutically acceptable auxiliary additives, as such (a) an inorganic or organic acid; (b) one or a mixture of preserving agents; (c) an amino acid co-formulator; wherein either the two combined, essential components (1) and (2) are either directly dissolved in water as a ready-to-use solution or wherein component (1) is prepared as solid powder to be reconstituted at the time of its use with a suitable volume of liquid, aqueous diluent or carrier.

Desirable physiologically active nasal peptides, which can be advantageously administered according to the present invention, are such peptides with a molecular weight ranging from 1000 to 150000 Dalton, in view of the fact that they are easily absorbed through the nasal mucous membrane. Especially those having a molecular weight ranging from 1000 to 50000 Dalton are more desirable. Such desirable physiologically active nasal peptides, which include also their pharmaceutically acceptable salts and their peptidic fragments, are exemplified in the following list, but to mention some of them, which shall be therefore not considered as a limitation.

For instance, such peptidic hormones and hormone derivatives as buserelin, desmopressin, vasopressin, angiotensin, felypressin, octreotide, somatropin, thyrotropin (TSH), somatostatin, gosereline, thryptorelin, insulin (obtained from caw and pig or synthetic or recombinant), protirelin, adrenocorticotropin (ACTH), prolactin, luteinizing hormone (LH), luteining hormone-release hormone (LH-RH), leuprorelin, calcitonin (human, chicken, eel, porcine or recombinant), carbocalcitonin, calcitonin gene related peptides (CGRP), kallikrein, parathyrin, glucagon, oxytocin, gastrin, secretin, leptin, nafarelin, serum gonadotropin, gonadotropin release factor, growth hormone, erytropoietin, hirudin, urograstrone, renin, human parathyroid hormone (h-PTH); such physiologically active proteins as lymphokine or monokine such as interferon and interleukin, transferrin, histaglobulin, macrocortine, endorphins, enkephalins, neurotensin; such as peptidic enzymes as lysozyme, urokinase, superoxide dismutase; such proteic vaccines as acellular and cellular pertussis vaccine, diphteria vaccine, tetanus vaccine, influenza vaccine; and such as peptidic toxoids as diphtheria toxoid, tetanus toxoid; can be suitably included in the pharmaceutical formulation of the invention.

Personalised proteins, a new category of medicinal product of peptidic nature derived from genoma, which can be personalized for each patient for a specific disease, can be also conveniently included.

Further according to the present invention the pharmaceutical formulation for nasal administration preferably comprises:
(1) a therapeutically effective amount of nasal peptide;
(2) the absorbefacient and stabilizer THAM;
   in a pharmaceutically acceptable liquid, aqueous diluent or carrier suitable for nasal application further comprising optionally other pharmaceutically acceptable auxiliary additives, such as (a) hydrochloric or citric acid; (b) one or a mixture of methyl or/and propyl p-hydroxybenzoate; (c) cysteine; wherein the two combined, essential components (1) and (2), when formulated as a ready-to-use solution together with the auxiliary additives, can be further put up in a mono-disposable or in a multidose delivery system device.

Preferred pharmaceutical formulations according to this invention comprise a combination of:
(1) a therapeutically effective amount of nasal peptide in concentrations of 0.001 microgram/ml to 50.0 mg/ml or of 10 Units/ml to 20000 Units/ml, which may conveniently vary for each selected nasal peptide according to the unitary therapeutic dose to be applied by intranasal route; and
(2) THAM in concentrations of 0.5 mg/ml to 30.0 mg/ml, which may conveniently vary for each selected nasal peptide in relation to the required absorbefacient activity and stability requirements;
   in a pharmaceutically acceptable liquid, aqueous diluent or carrier comprising optionally other pharmaceutically acceptable auxiliary additives, such as (a) hydrochloric acid 0.1 N in concentrations of 0.3 mg/ml to 30.0 mg/ml or citric acid in concentrations of 0.6 mg/ml to 60.0 mg/ml; (b) one or a mixture of methyl or/and propyl p-hydroxybenzoate in concentrations of 0.1 mg/ml to 3.0 mg/ml; (c) cysteine in concentrations of 0.05 mg/ml to 50.0 mg/ml.

It is particularly preferred that:
(1) the therapeutically effective dose of a nasal peptide is in concentrations of 0.01 microgram/ml to 10.0 mg/ml or of 20 Units/ml to 12500 Units/ml;
(2) THAM is in concentrations of 2.0 mg/ml to 4.5 mg/ml;
   and that the pharmaceutically acceptable liquid, aqueous diluent or carrier further comprises optionally other pharmaceutically acceptable auxiliary additives, such as (a) citric acid monohydrate in concentrations of 2.8 mg/ml to 6.2 mg/ml; (b) a mixture of methyl and propyl p-hydroxybenzoate not totally exceeding 0.3 mg/ml, but with a ratio of 2:1 to 20:1; (c) cysteine in concentrations of 0.5 mg/ml to 10.0 mg/ml.

Moreover it has been surprisingly found that THAM, when combined in the pharmaceutical formulation comprising a nasal peptide, enhances the absorbefacient properties of the pharmaceutical formulation and the nasal peptide bioavailability levels consequential to nasal application.

In fact it has been unexpectedly noticed that one of the most striking characteristics of THAM in the instant invention is that this organic hydrogen-ion acceptor produces a marked biological activity in vivo and physiologically and reversibly depolarizes the nasal mucosa epithelial cell membranes, thus enhancing the active process of nasal peptide absorption. Furthermore TRAM, contrarily to other amines, produces such desirable effects at concentrations where other amines exhibit significant toxicity problems.

A further surprising technical advantage of the instant invention is that THAM significantly contributes to the stabilization of the nasal peptide comprised in the pharmaceutical formulation. In fact it has been surprisingly observed that liquid pharmaceutical formulations containing THAM do not easily absorb O2 and CO2 from the atmosphere, thus avoiding the contact with oxygen and improving the stability profile during production, storage and use, so that the production under nitrogen flow is also an optional choice.

In other words, it has been experimentally observed that TRAM prevents the oxidation of the disulphide bridges between the thioamino acids of the nasal peptides, thus unexpectedly stabilizing the therapeutically effective amount of the nasal peptide of the pharmaceutical formulation.

In accordance with the instant invention a method of producing the pharmaceutical formulation is also provided. The pharmaceutical formulation, comprising the combination of the selected nasal peptide or of its salt or peptidic fragment and of absorbefacient and stabilizer THAM in a liquid diluent or carrier, said aqueous solution comprising optionally other pharmaceutically acceptable auxiliary additives, is manufactured through a method, which is substantially different when a ready-to-use (mono-disposable or multidose) or a reconstituted (multidose) solution is desired. Therefore the method of producing the pharmaceutical formulation comprises, for example, basic steps, which are summarized by way of example below.

(A) Ready-to-Use Solution:

(a1) In a convenient amount of distilled water is dissolved in a suitable container the adequate quantity of TRAM and optionally of methyl or/and propyl p-hydroxybenzoate, hydrochloric or citric acid, cysteine and stirred until complete dissolution;
(a2) the adequate quantity of pharmaceutically active nasal peptide or its salt or fragment is then thoroughly dissolved in solution (al), stirring gently in order to avoid foaming.

The method of producing further comprises the steps of:
(a3) filtering solution (a2) for sterilization thereof; and
(a4) filling a mono-disposable or a multidose container with the desired quantity of filtrate. The monodose is an integrated system, but the multidose container has to be conveniently sealed with a delivery system device for nasal administration, as it is described later on.

The above delivery system device dispenses at every actuation the determined volume (equivalent to a therapeutically effective unit dose) of nasal peptide solution. The ready-to-use solution is preferred for those nasal peptides showing a satisfactory shelf life profile, even when formulated in aqueous solutions, during the storage period before their use and also during the in-use period after opening.

The nasal delivery system device, suitably produced and already available on the market for this purpose, may contain sufficient pharmaceutical formulation for dispensing a single nasal dose unit or several sequential unit doses (hence the term “multidose”) over a period of days or weeks. The delivered quantity (metered volume). corresponds to a therapeutically effective dose unit of nasal peptide to be applied to the mucus-lined epithelium of the nasal mucosa, as already determined for each selected nasal peptide.

Thus, in accordance with the foregoing, the present invention further provides a convenient delivery system device for nasal administration of a therapeutically effective amount of the nasal peptide of the pharmaceutical formulation, which comprises a suitable container, a metered precision pump delivering the exact metered volume of solution, the nasal applicator enabling the administration in the form of a drop type or of spray to the nasal epithelium, said convenient delivery system device comprising a combination of:
(1) a therapeutically effective amount of physiologically active nasal peptide or its salt or fragment, as therapeutically active ingredient; and of
(2) the absorbefacient and stabilizer THAM;
   in a liquid, aqueous diluent or carrier, said solution comprising optionally other pharmaceutically acceptable auxiliary additives, such as (a) an inorganic or organic acid; (b) one or a mixture of methyl or/and propyl p-hydroxybenzoate; (c) an amino acid co-formulator;

Therefore the instant invention provides as well as a method of administering a therapeutically effective amount of nasal peptide or its salt or fragment to a patient requiring nasal peptide treatment, which method comprises administering the pharmaceutical formulation, as previously defined, to said patient via the nasal route.

The container, the metered precision pump and the nasal applicator may be integrated also as a unit just dispensing one dose only and it can also be made disposable.

The delivery system device of a multidose dispenser may be also equipped with dose counting system.

(B) Reconstituted Solution (Containers n.° 1 and n.° 2)

(b1) The adequate amount of nasal peptide or its salt or fragment is dissolved separately in a suitable quantity of solvent; after filtering the solution and filling suitably a multidose container with the predetermined volume of filtrate, the solution is conventionally lyophilized; the container is then suitably sealed by means of one of the stopper systems available on the market for this purpose (container n.° 1—nasal peptide powder);
(b2) In a convenient amount of distilled water is dissolved in a suitable container the adequate quantity of THAM and optionally of methyl or/and propyl p-hydroxybenzoate, hydrochloric or citric acid, cysteine and stirred until complete dissolution; the resulting solution is filtered for sterilization thereof; the desired volume of the filtrate is filled in suitable containers and conveniently sealed by means of one of the stopper systems available on the market for this purpose (container n.° 2—solvent mixture for reconstitution).

Therefore, at the time of starting its use, the nasal peptide powder is reconstituted as nasal solution by pouring the solvent mixture of container n.° 2 into container n.° 1 and mixing thoroughly by rotating the container until complete dissolution of the nasal peptide powder. Thereafter a nasal device system, as described for the prior section (A) Ready-to-use solution, but suitably equipped with a screw precision pump, is conveniently installed on the neck with screw closure of container n.° 1. The reconstituted solution is preferred for those nasal peptides showing a not satisfactory shelf life profile, when formulated in aqueous solutions during the storage period before its use.

Both the ready-to-use solution and the reconstituted solution (containers n.° 1 and n.° 2) are preserved at suitable storage conditions, in accordance with their stability results, and in most cases, the storage controlled temperature shall be in the range of +5°±3° C., while in other cases the storage temperature shall not exceed +25°±2° C.

According to the present invention, the therapeutically effective dose of the nasal peptide or of its salt or fragment contained in the pharmaceutical formulation for administration through the mucus-lined epithelium of the nasal mucosa may vary basically with the kind of selected nasal peptide or its salt or fragment and also with patient's age, body weight, severity of disease, desired therapeutic response, health conditions and other drugs simultaneously administered. Generally, the dose of the pharmaceutical formulation for the nasal administration of the present invention, which contains a pharmaceutically active nasal peptide, may be determined according to the known administered doses of the used nasal peptide.

The invention will now be explained in detail by reference to the following experimental examples, which are described only for presentation of further details rather than for limiting the scope of the invention itself.

### EXAMPLE 1

### Long Shelf Life and In-Use Stable Multidose Formulation of Desmorpressin

This Example relates to a multidose nasal spray pharmaceutical formulation according to the present invention (Formulation (A)]. This Formulation has a shelf life of more than two years when stored at controlled refrigerated conditions (t°: +5°±3° C.), and a shelf life of one month after opening, when stored at room temperature (t°: +25°±2° C.). The Formulation has the following composition:

Formulation (A)

| | | | |
|---|---|---|---|
| 1a) | desmopressin acetate (DDAVP) | 112.60 | mg |
| | (equivalent to desmopressin | 100.00 | mg) |
| 2a) | THAM | 4.40 | g |
| 3a) | citric acid | 6.28 | g |
| 4a) | methyl p-hydroxybenzoate | 0.27 | g |
| 5a) | propyl p-hydroxybenzoate | 0.03 | g |
| 6a) | distilled water q.s. to | 1000.00 | ml |

Formulation (A) was compared to a preparation already on the market (Formulation (B)], having the following declared composition: Formulation B

Formulation B

| | | | |
|---|---|---|---|
| 1b) | desmopressin acetate (DDAVP) | 100.00 | mg |
| 2b) | sodium chloride | 7.50 | g |
| 3b) | citric acid monohydrate | 1.70 | g |
| 4b) | disodium phosphate dihydrate | 3.00 | g |
| 5b) | benzalkonium chloride solution (50%) | 0.20 | g |
| 6b) | sterile water q.s. to | 1000 | ml |

The stability results are summarized in Tables n.° 1 and n.° 2.

The stability profile of Formulation (A) at different testing intervals of both studies is equivalent or even a little better than the reference product already on the market.

TABLE n.° 1
Comparative results of the assay of two desmopressin Formulations (A)
and (B) at different testing intervals during 24 months storage at controlled
refrigerated conditions (+5° ± 3° C.)

| Testing intervals | Formulation (A) | Formulation (B) |
|---|---|---|
| (months = m) | Desmopressin | Desmopressin |
| Storage: +5° ± 3° C. | Assay % | Assay % |
| time 0 | 100.0 | 100.0 |
| 3 m | 99.7 | 99.5 |
| 6 m | 99.2 | 99.0 |
| 9 m | 98.3 | 97.9 |
| 12 m | 97.9 | 97.3 |
| 18 m | 97.2 | 96.1 |
| 24 m | 96.5 | 95.7 |
| 30 m | 95.2 | 94.4 |
| Difference 0-30 m | 4.8 | 5.6 |
| Difference 0-30 m | 100.0% | 85.7% |
| A (100%)/B | | (−14.3%) |

TABLE n.° 2
Comparative results of the assay of two desmopressin Formulations
(A) and (B) at different testing intervals during 30 days in-use
conditions after opening with subsequent storage at constant
room temperature (+25° ± 2° C.)

| Testing intervals | Formulation (A) | Formulation (B) |
|---|---|---|
| (days = d) | Desmopressin | Desmopressin |
| Storage: +25° ± 2° C. | Assay % | Assay % |
| time 0 | 100.0 | 100.0 |
| 7 d | 99.4 | 99.3 |
| 15 d | 98.3 | 98.1 |
| 30 d | 97.5 | 96.8 |
| Difference 0-30 d | 2.5 | 3.2 |
| Difference 0-30 d | 100.0% | 78.1% |
| A (100%)/B | | (−21.9%) |

### EXAMPLE 2

### Long Shelf and In-Use Stable Multidose Formulation of Buserlin

This Example relates to a multidose nasal spray pharmaceutical formulation of buserelin according to the present invention (Formulation (C)]. The Formulation has a shelf life of more than two years when stored at controlled refrigerated conditions (t°: +5°±3° C.), and an in-use shelf life of one month after opening, when stored at room temperature (t°: +25°±2° C.). The Formulation has the following composition.

Formulation (C)

| | | | |
|---|---|---|---|
| 1c) | buserelin acetate | 10.50 | mg |
| | (equivalent to buserelin | 10.00 | mg) |
| 2c) | THAM | 42.00 | mg |
| 3c) | citric acid | 60.00 | mg |
| 4c) | methyl p-hydroxybenzoate | 2.70 | mg |
| 5c) | propyl p-hydroxybenzoate | 0.30 | mg |
| 6c) | distilled water q.s. to | 10.00 | g |

This Formulation was compared with a preparation already on the market [Formulation (D)], having the following declared composition:

Formulation (D)

| | | | |
|---|---|---|---|
| 1d) | buserelin acetate | 10.50 | mg |
| | (equivalent to buserelin | 10.00 | mg) |
| 2d) | sodium chloride | 80.0 | mg |
| 3d) | sodium citrate | 24.00 | mg |
| 4d) | citric acid monohydrate | 4.00 | mg |
| 5d) | benzalkonium chloride | 1.00 | mg |
| 6d) | water for injections | 10.00 | g |

The stability results are summarized in Tables n.° 3 and n.° 4.

The stability profile of Formulation (C) at different testing intervals of both studies is equivalent or even a little better than the reference product already on the market.

TABLE n.° 3
Comparative results of the assay of two buserelin Formulations (C)
and (D) at different testing intervals during 24 months storage at
controlled refrigerated conditions (+5° ± 3° C.)

| Testing intervals | Formulation (C) | Formulation (D) |
|---|---|---|
| (months = m) | Buserelin | Buserelin |
| Storage: +5° ± 3° C. | Assay % | Assay % |
| time 0 | 100.0 | 100.0 |
| 3 m | 99.8 | 99.7 |
| 6 m | 99.4 | 99.3 |
| 9 m | 98.7 | 98.2 |
| 12 m | 98.4 | 97.8 |
| 18 m | 97.9 | 96.5 |
| 24 m | 97.1 | 96.8 |
| 30 m | 96.4 | 94.7 |
| Difference 0-30 m | 3.6 | 5.3 |
| Difference 0-30 m | 100.0% | 64.1% |
| C (100%)/D | | (−35.9%) |

TABLE n° 4
Comparative results of the assay of two buserelin formulations
(C) and (□D) at different testing intervals during 30 days
in-use conditions after opening with subsequent storage at constant
room temperature (+25° ± 2° C.)

| Testing intervals | Formulation (C) | Formulation (D) |
|---|---|---|
| (days = d) | Buserelin | Buserelin |
| Storage: +25° ± 2° C. | Assay % | Assay % |
| time 0 | 100.0 | 100.0 |
| 7 d | 99.5 | 99.5 |
| 15 d | 98.8 | 98.4 |
| 30 d | 97.9 | 97.5 |
| Difference 0-30 d | 2.1 | 2.5 |
| Difference 0-30 d | 100.0% | 84.0% |
| C (100%)/D | | (−26.0%) |

### EXAMPLE 3

### Pharmaceutical Formulation for Nasal Administration Containing Desmopressin [Test Formulation (A) of Example 1] and Its Method of Preparation

In this Example, a nasal spray pharmaceutical formulation of desmopressin of Formulation (A) (see Example 1) was prepared as a ready-to-use solution. Ingredients were used in a scale volume to produce final volume of 1000.0 ml (corresponding to about 400 units). First ingredients 4a) and 5a) were dissolved in about 800.0 ml of 6a) to complete dissolution. Thereafter 2a) and 3a) were added by mixing thoroughly. When dissolution was completed, la) was added by mixing carefully to avoid foaming and the remaining 200.0 ml of 6a) were added to yield 1000.0 ml solution. The obtained solution was filtered (e.g. using a 0.2 micron filter Pall brand) to yield a composition suitable for nasal application. The filtered solution was introduced into individual nasal spray multidose containers, each with a solution volume of 2.5 ml. The filling step was carried out in a bacteriologically controlled area of, for example, class 100 or 1000. The composition comprises a total of 0.25 mg of active ingredient and the metered pump system was suitable to deliver subsequent individual doses of 100 microliters (e.g. 10 micrograms of desmopressin per actuation).

Similarly, by using the same formulation, metered dosing system and production techniques, but half amount of ingredient 1a), it was possible to obtain a ready-to-use solution delivering 5 micrograms of desmopressin per 100 microliters actuation.

### EXAMPLE 4

### Pharmaceutical Formulation for Nasal Administration Containing Buserlin [Test Formulation (C) of Example 2] and Its Method of Preparation

In this Example, a nasal spray pharmaceutical formulation of buserelin of Formulation (C) (see Example 2) was prepared as ready-to-use solution.

Ingredients were used in a scale volume to produce a final volume of 10.0 litres (corresponding to about 1000 units). Ingredients 4c) and 5c) were dissolved in an aliquot of 8.0 litres of 6c) and after complete dissolution ingredients 2c) and 3c) were added and mixed thoroughly. Finally, 1c) was added to complete dissolution. The resulting solution was filtered by means of a 0.2 micron filter (Pall brand) to give a composition suitable for nasal application. The filtered solution was introduced into nasal spray multidose dispensers, each with a solution weight of 10.0 grams. The filling step was carried out in a bacteriologically controlled area, of, for example, class 100 or 1000. Each container comprises 10.0 mg of buserelin/10.0 g solution and the nasal applicator delivers subsequent individual doses of 0.1 mg buserelin/100 microliters volume per actuation.

### EXAMPLE 5

### Pharmaceutical Formualtion Containing Insulin Suitable for Nasal Administration and Its Method of Preparation

A pharmaceutical formulation of insulin (Formulation (E)], in the form type of nasal spray having the following composition, was prepared as reconstituted solution:

* Preparation of container n.° 1 (powder):

| | |
|---|---|
| 1e) insulin | 5000 Units |

* Preparation of container n.° 2 (solvent mixture):

| | | | |
|---|---|---|---|
| 2e) | THAM | 58.0 | mg |
| 3e) | hydrochloric acid 0.1 N | 29.0 | mg |
| 4e) | methyl p-hydroxybenzoate | 12.0 | mg |
| 5e) | distilled water q.s. to | 10.00 | ml |

Ingredients were used in a scale volume to produce a total of 100 containers of each type. Container n.° 1 was either prepared by dosing directly in the container the corresponding weight of powder 1e) or by preparing a suitable solution with a known concentration of 1e), pouring the individually dosed volume directly into the container and then lyophilizing it directly in the container to yield the lyophilized powder. The solvent mixture of container n.° 2 was prepared by dissolving in 5e) the ingredients 2e), 3e) and 4e) with the same sequence and techniques of Example 3, but adjusting before filtration the pH to 7.0-7.2 by means either of 2e) or 3e). The resulting solvent mixture, suitable for nasal administration, was used for filling, in a bacteriologically controlled area class 100 or 1000, in containers n.° 2, each dosed at 10.0 ml volume.

Both containers were conveniently sealed with suitable stoppers already available on the market for this purpose.

The insulin powder of container n.° 1 may be reconstituted at the time of its use by pouring in container n.° 1 the solvent mixture of container n.° 2. The selected metered pump with screw system, equipped with nasal applicator and relevant cap, was then installed on the screw neck of container n.° 1, already containing the reconstituted nasal solution.

The total volume of each reconstituted multidose container was 10.0 ml (5000 Units of insulin/ml), while the dosing system was suitable to deliver subsequent individual doses, each containing 200 microliters of solution, equivalent to 100 Units of insulin per actuation.

### EXAMPLE 6

### Pharmaceutical Formulation Containing h-PTH (1-34) Suitable for Nasal Administration and Its Method of Preparation

A nasal spray pharmaceutical formulation of human-PTH (1-34)

[Formulation (F)] having the following composition:

| | | | |
|---|---|---|---|
| 1f) | h-PTH (1-34) | 1000 | Units |
| 2f) | THAM | 41.50 | mg |
| 3f) | citric acid | 60.50 | mg |
| 4f) | methyl p-hydroxybenzoate | 2.50 | mg |
| 5f) | propyl p-hydroxybenzoate | 0.30 | mg |
| 6f) | distilled water q.s. to | 1.00 | ml |

was prepared as ready-to-use solution.

Ingredients were used in a scale volume to produce a final volume of 1.0 litre (corresponding to about 300 units of 3.0 ml each).

Ingredients 9f) and 5f) were dissolved in an aliquot of 800.0 ml of 6f) and, after complete dissolution, ingredients 2f) and 3f) were added and mixed thoroughly. Finally, if) was added to complete dissolution and the remaining 200.0 ml of 6f) were added to yield 1.0 litre solution. The resulting solution was filtered by means of a 0.2 micron filter (Pall brand) to yield a composition suitable for nasal application. The filtered solution was used for filling, in a bacteriologically controlled area of, for example, class 100 or 1000, nasal spray multidose dispensers with a solution volume of 3.0 ml. Each container comprises 3000 Units of h-PTH/3.0 ml of solution and the metered pump system was suitable to deliver subsequent individual doses of 100 Units of h-PTH/100 microliters volume per each actuation.

Similarly, by using the same formulation, metered dosing system and production techniques, but using double amount (2000 Units) and four times (4000 Units) of the ingredient 1f), it was possible to obtain a ready-to-use solution delivering 200 Units and 400 Units of h-PTH/100 microliters volume of each actuation respectively.

### EXAMPLE 7

### Relative Bioavailability Study of Two Carbocalcitonin Formulations for Nasal Administration

Formulation (G) of the invention, containing carbocalcitonin, was compared in a pilot study with Formulation (H), a composition already available on the market and prepared according to the prior known art. Particularly the pharmacokinetic parameters following to nasal administration of the same dosage (one single dose of 40 MRC in 100 microliters nasal solution/actuation) of carbocalcitonin contained in the two Formulations (G) and (H) in 12 subjects (administration sequence at random, wash out interval between the two dosages of 48 hours, determination of carbocalcitonin plasma concentrations by radioimmunoassay (RIA) method and reagents known in the art and statistical elaboration of the resulting biodynamic parameters) were determined.

The composition of each formulation is as follows.

Formulation (G)
1.0 ml of ready-to-use nasal solution (G) containing:

| | | | |
|---|---|---|---|
| 1g) | carbocalcitonin | 400 | MRC |
| 2g) | THAM | 4.20 | mg |
| 3g) | citric acid | 6.00 | mg |
| 4g) | methyl p-hydroxybenzoate | 1.00 | mg |
| 5g) | propyl p-hydroxybenzoate | 0.10 | mg |
| 6g) | distilled water q.s. to | 1.00 | ml |

Formulation (H)
1.0 ml of ready-to-use nasal solution (H) containing:

| | | | |
|---|---|---|---|
| 1h) | carbocalcitonin | 400 | MRC |
| 2h) | ammonium glycyrrhizinate | 20.0 | mg |
| 3h) | sodium chloride | 6.00 | mg |
| 4h) | sodium citrate | 4.63 | mg |
| 5h) | citric acid anhydrous | 0.37 | mg |
| 6h) | methyl p-hydroxybenzoate | 1.30 | mg |
| 7h) | propyl p-hydroxybenzoate | 0.20 | mg |
| 8h) | depurated water q.s. to | 1.00 | ml |

Ingredients of Formulation (G) were used in a scale volume to produce a pilot batch of 200 units. The ready-to-use solution (G) was prepared according to the method already disclosed in Example 3, while Formulation (H) was already available on the market.

The most important pharmacokinetic parameters of plasma concentrations in the 12 subjects at different intervals following to a nasal administration of an individual single dose of 40 MRC of carbocalcitonin (separately of Formulation (G) and Formulation (H), each delivered by means of a multidose container dispensing 100 microliters volume) were statistically expressed as Cmax (highest observed concentration), Tmax (time of highest observed concentration), AUCtot (total area under concentration curve) and T1/2 (half-life time). They are reported in the following Tables n. 5 and 6.

TABLE n.° 5
Formulation (G) - Pharmacokinetic parameters of plasma
concentrations from a relative, pilot bioavailability
study in 12 subjects following to a nasal administration
of a single dose of 40 MRC of carbocalcitonin solution
(G) (100 microliters volume of nasal solution).

| Subject n. | Cmax | Tmax | AUCtot | T ½ |
|---|---|---|---|---|
| 1A | 192.91 | 16.00 | 2851.21 | 5.23 |
| 2A | 151.93 | 9.00 | 2374.02 | 7.74 |
| 3A | 190.08 | 17.00 | 2872.65 | 6.19 |
| 4A | 165.25 | 13.00 | 3144.23 | 5.52 |
| 5A | 172.71 | 14.00 | 2563.96 | 6.04 |
| 6A | 098.37 | 17.00 | 2135.80 | 7.38 |
| 7A | 193.14 | 13.00 | 2592.17 | 4.03 |
| 8A | 167.87 | 16.00 | 3261.35 | 6.75 |
| 9A | 200.89 | 17.00 | 3770.24 | 7.02 |
| 10A | 188.76 | 11.00 | 3445.18 | 10.03 |
| 11A | 138.86 | 15.00 | 2144.09 | 3.98 |
| 12A | 135.38 | 14.00 | 2062.46 | 6.12 |
| Min | 98.37 | 9.00 | 2062.46 | 4.03 |
| Max | 200.89 | 17.00 | 3445.18 | 9.81 |
| N | 12 | 12 | 12 | 12 |
| Mean | 166.35 | 14.33 | 2768.11 | 6.34 |

TABLE n.° 6
Formulation (H) - Pharmacokinetic parameters of plasma
concentrations from a relative, pilot bioavailability
study in 12 subjects following to a nasal administration
of a single dose of 40 MRC of carbocalcitonin solution
(H) (100 microliters volume of nasal solution).

| Subject n. | Cmax | Tmax | AUCtot | T ½ |
|---|---|---|---|---|
| 1B | 111.66 | 15.00 | 2161.33 | 7.09 |
| 2B | 122.32 | 13.00 | 2379.76 | 4.74 |
| 3B | 179.71 | 17.00 | 3250.51 | 6.43 |
| 4B | 155.35 | 13.00 | 3114.19 | 6.32 |
| 5B | 158.95 | 14.00 | 2441.47 | 5.93 |
| 6B | 126.76 | 13.00 | 1969.03 | 3.99 |
| 7B | 128.91 | 15.00 | 2087.82 | 5.60 |
| 8B | 181.16 | 17.00 | 3398.76 | 6.53 |
| 9B | 138.70 | 9.00 | 2484.05 | 7.48 |
| 10B | 173.22 | 17.00 | 2918.30 | 4.11 |
| 11B | 105.37 | 15.00 | 1869.24 | 4.99 |
| 12B | 108.88 | 13.00 | 2168.93 | 5.07 |
| Min | 105.3700 | 9.00 | 1869.2400 | 3.9900 |
| Max | 181.1600 | 17.00 | 3398.7600 | 7.4800 |
| N | 12 | 12 | 12 | 12 |
| Mean | 140.92 | 14.25 | 2520.28 | 5.69 |

The differences between the most significant biodynamic parameters of the two Formulations (G) and (H) are slightly in favour of Formulation (G). However, they are not statistically significant. Therefore the two tested formulations shall be regarded as bioequivalent.

### EXAMPLE 8

### Relative Bioavailibility Study of Two Calcitonin (Salmon) Nasal Spray Formulations

Formulation (I) of the invention, containing calcitonin (salmon), was compared in a pilot study with Formulation (K), a composition already available on the market and prepared according to the prior known art. Particularly, the pharmacokinetic parameters following to nasal administration of the same dosage (one single dose of 200 MRC in 90 microliters nasal solution/actuation) of calcitonin (salmon) contained in the two different Formulations (I) and (K) in 12 subjects (administration sequence at random, wash out interval between the two dosages of 72 hours, determination of calcitonin (salmon) plasma concentrations by radioimmunoassay (RIA) with method and reagents known in the art and statistical elaboration of the resulting biodynamic parameters) were determined.

The compositions of each formulation are as follows.

Formulation (I)
1.0 ml of ready-to-use nasal solution (I) containing:

| | | | |
|---|---|---|---|
| 1i) | calcitonin (salmon) | 2200 | MRC |
| 2i) | THAM | 4.20 | mg |
| 3i) | citric acid | 6.00 | mg |
| 4i) | distilled water q.s. to | 1.00 | ml |

Formulation (K)
1.0 ml of ready-to-use nasal solution (K) containing:

| 1k) | calcitonin (salmon) | 2200 | MRC |
|---|---|---|---|
| 2k) | sodium chloride | 8.50 | mg |
| 3k) | benzalkonium chloride | 0.10 | mg |
| 4k) | hydrochloric acid (1.0 N) | to adjust pH | |
| 5k) | nitrogen gas | to replace air | |
| 6k) | purified water q.s. to | 1.00 | ml |

Ingredients of Formulation (I) were used in a scale volume to produce a pilot batch of 200 units. The ready-to-use solution (I) was prepared according to the method already disclosed in Example 3, except that solution (I) was filled in aseptic conditions in a container equipped with a special sterile device system, purposely manufactured and available on the market, having an additional small cylindrical filter which conveniently sterilizes the atmospheric air entering into the container after each actuation to compensate the inner depression.

Formulation (K) was already available on the market.

The most important pharmacokinetic parameters of plasma concentrations in the 12 subjects at different intervals following to a nasal administration of an individual single dose of 200 MRC of calcitonin (salmon) (separately of Formulation (I) and Formulation (K), each delivered by means of a multidose container dispensing 90 microliters volume) were statistically expressed as Cmax (highest observed concentration), Tmax (time of highest observed concentration), AUCtot (total area under concentration curve) and T1/2 (half-life time). They are reported in the following Tables n. 7 and 8.

TABLE n.° 7
Formulation (I) - Pharmacokinetic parameters of plasma
concentrations from a relative, pilot bioavailability study
in 12 subjects following to a nasal administration of a
single dose of 200 MRC of calcitonin (salmon) solution
(I) (90 microliters volume of nasal solution).

| Subject n. | Cmax | Tmax | AUCtot | T ½ |
|---|---|---|---|---|
| 1C | 81.34 | 16.00 | 3364.37 | 37.36 |
| 2C | 84.58 | 25.00 | 4200.41 | 46.34 |
| 3C | 77.57 | 15.00 | 3182.16 | 38.05 |
| 4C | 76.16 | 15.00 | 2851.65 | 37.42 |
| 5C | 76.48 | 16.00 | 2897.98 | 37.44 |
| 6C | 83.75 | 20.00 | 4152.29 | 51.12 |
| 7C | 81.13 | 24.00 | 3331.42 | 47.37 |
| 8C | 77.51 | 19.00 | 3537.83 | 51.74 |
| 9C | 69.12 | 25.00 | 3542.56 | 52.44 |
| 10C | 83.70 | 20.00 | 3907.19 | 50.91 |
| 11C | 85.29 | 19.00 | 4215.78 | 55.98 |
| 12C | 82.05 | 21.00 | 3940.03 | 53.23 |
| Min | 69.12 | 15.00 | 2851.65 | 37.36 |
| Max | 85.26 | 25.00 | 4215.78 | 55.98 |
| N | 12 | 12 | 12 | 12 |
| Mean | 79.89 | 19.58 | 3593.64 | 46.62 |

TABLE n.° 8
Formulation (K) - Pharmacokinetic parameters of plasma
concentrations from a relative, pilot bioavailability study
in 12 subjects following to a nasal administration of a
single dose of 200 MRC of calcitonin (salmon) solution
(K) (90 microliters volume of nasal solution).

| Subject n. | Cmax | Tmax | AUCtot | T ½ |
|---|---|---|---|---|
| 1D | 74.04 | 16.00 | 2621.47 | 43.05 |
| 2D | 75.56 | 25.00 | 3653.31 | 51.69 |
| 3D | 71.29 | 19.00 | 2847.96 | 43.55 |
| 4D | 70.76 | 20.00 | 2781.60 | 53.81 |
| 5D | 69.03 | 25.00 | 3121.25 | 45.92 |
| 6D | 73.59 | 24.00 | 3407.59 | 48.37 |
| 7D | 67.90 | 18.00 | 2372.33 | 39.71 |
| 8D | 74.45 | 15.00 | 2921.84 | 39.78 |
| 9D | 67.87 | 23.00 | 3147.82 | 42.40 |
| 10D | 82.09 | 22.00 | 3991.74 | 50.38 |
| 11D | 75.32 | 15.00 | 2974.38 | 40.39 |
| 12D | 78.41 | 21.00 | 3779.15 | 54.92 |
| Min | 67.87 | 15.00 | 2372.33 | 39.71 |
| Max | 82.09 | 25.00 | 3991.74 | 54.92 |
| N | 12 | 12 | 12 | 12 |
| Mean | 73.36 | 20.25 | 3135.04 | 46.16 |

The differences between the most significant biodynamic parameters of the two Formulations (I) and (K) are slightly in favour of Formulation (I), despite they are not so statistically significant, and therefore the two tested formulations shall be regarded as bioequivalent.

While the various embodiments of the present invention have been described herein, it is possible that a skilled artisan could modify the combination of the basic and optional ingredients and the production conditions and obtain similar or equivalent results. Such modifications are contemplated as being within the scope of the present disclosure.

1. A pharmaceutical formulation comprising:
(1) THAM [tris(hydroxymethyl) aminomethane] as a selective absorbefacient to enhance through the nasal mucosa-lined-epithelium the absorption of substances of peptide nature; and
(2) a therapeutically effective amount of active nasal peptide, its pharmaceutically acceptable salt or its peptidic fragment;
in a pharmaceutically acceptable, aqueous liquid diluent or carrier, said formulation being in a form suitable for nasal administration.

2. The pharmaceutical formulation, according to claim 1, wherein the nasal peptide, its pharmaceutically acceptable salt or its peptidic fragment is selected from the group consisting of peptide hormones and hormone derivatives, physiologically active lymphokines and monokines, peptidic enzymes, proteic vaccines, peptidic toxoids, and personalized proteins derived from genoma, in a form suitable for nasal administration.

3. The pharmaceutical formulation, according to claim 1, wherein the nasal peptide, its pharmaceutically acceptable salt or its peptidic fragment is selected from the group consisting of the peptide hormones and hormone derivatives buserelin, desmopressin, vasopressin, angiotensin, felypressin, octreotide, somatropin, thyrotropin (TSH), somatostatin, gosereline, thryptorelin and insulin (selected from the group consisting of caw and pig, synthetic, and recombinant).

4. The pharmaceutical formulation, according to claim 1, wherein the nasal peptide, its pharmaceutically acceptable salt or its peptidic fragment is selected from the group consisting of the peptide hormones and hormone derivatives protirelin, adrenocorticotropin (ACTH), prolactin, luteinizing hormone (LH), luteinizing hormone-release hormone (LH-RH), leuprorelin, calcitonin (selected from the group consisting of human, chicken, eel, porcine and recombinant), carbocalcitonin and calcitonin gene related peptides (CGRP).

5. The pharmaceutical formulation, according to claim 1, wherein the nasal peptide, its pharmaceutically acceptable salt or its peptidic fragment is selected from the group consisting of the peptide hormones and hormone derivatives kallikrein, parathyrin, glucagon, oxytocin, gastrin, secretin, leptin, nafarelin, serum gonadotropin, gonadotropin release factor, growth hormone, erytropoietin, hirudin, urograstrone, renin and human parathyroid hormone (h-PTH).

6. The pharmaceutical formulation, according to claim 1, wherein the nasal peptide, its pharmaceutically acceptable salt or its peptidic fragment is selected from the group consisting of the physiologically active lymphokines and monokines interferon, interleukin, transferrin, histaglobulin, macrocortine, endorphins, enkephalins and neurotensin.

7. The pharmaceutical formulation, according to claim 1, wherein the nasal peptide, its pharmaceutically acceptable salt or its peptidic fragment is selected from the group consisting of the peptidic enzymes lysozyme, urokinase and superoxide dismutase.

8. The pharmaceutical formulation, according to claim 1, wherein the nasal peptide, its pharmaceutically acceptable salt or its peptidic fragment is selected from the group consisting of the proteic vaccines as acellular and cellular pertussis, diphtheria, tetanus and influenza vaccines.

9. The pharmaceutical formulation, according to claim 1, wherein the nasal peptide, its pharmaceutically acceptable salt or its peptidic fragment is selected from the group consisting of the peptidic toxoids diphtheria, and tetanus and the personalized proteins derived from genoma.

10. The pharmaceutical formulation, according to claim 1, wherein (1) the therapeutically effective amount of active nasal peptide, its pharmaceutically acceptable salt or its peptidic fragment is in a concentration of 0.001. microgram/ml to 50.0 mg/ml or of 10 Units/ml to 20000 Units/ml, in relation to the therapeutically effective dose for administration by the endonasal route; and (2) THAM is in a combination of 0.5 mg/ml to 30.0 mg/ml.

11. The pharmaceutical formulation, according to claim 1, wherein (1) the therapeutically effective amount of active nasal peptide, its pharmaceutically acceptable salt or its peptidic fragment is in a concentration of 0.01 microgram/ml to 50.0 mg/ml or of 20 Units/ml to 12500 Units/ml; and (2) THAM is in a concentration of 2.0 mg/ml to 10.0 mg/ml.

12. The pharmaceutical formulation, according to claim 1, wherein (1) the therapeutically effective amount of active nasal peptide, its pharmaceutically acceptable salt or its peptidic fragment is in a concentration of 0.05 microgram/ml to 10.0 mg/ml or of 100 Units/ml to 6000 Units/ml; and (2) THAM is in a concentration of 2.5 mg/ml to 4.5 mg/ml.

13. The pharmaceutical formulation, according to claim 1, wherein said pharmaceutical formulation is in the form of ready-to-use or of reconstituted solution suitable for nasal administration in the form of a drop type or of a nasal spray.

14. The pharmaceutical formulation, according to claim 1, for administration in a metered single dose volume or in multiple doses thereof, each actuation comprising a metered dose volume between 50 microliters and 200 microliters.

15. A method for producing a pharmaceutical formulation according to claim 1, wherein the aqueous liquid diluent or carrier comprises the pharmaceutically acceptable auxiliary as additive (a) hydrochloric or citric acid; (b) one or a mixture of methyl or/and propyl p-hydroxybenzoate; and/or (c) cysteine.

16. The method according to claim 15, wherein the pharmaceutically acceptable, aqueous liquid diluent or carrier further comprises the pharmaceutically acceptable additive (a) hydrochloric acid 0.1 N in a concentration of 0.3 mg/ml to 50.0 mg/ml or citric acid in a concentration of 0.6 mg/ml to 60.0 mg/ml; (b) one or a mixture of methyl or/and propyl p-hydroxybenzoate in a concentration not exceeding 0.3 mg/ml with a ratio of 2:1 to 20:1; and (c) cysteine in a concentration of 0.5 mg/ml to 10.0 mg/ml.

17. A method for producing a pharmaceutical formulation for nasal administration according to claim 1, in the form of a ready-to-use solution, said method comprising the steps of: adding an adequate amount of distilled water to THAM, and optionally to methyl or/and propyl p-hydroxybenzoate, hydrochloric or citric acid and cysteine until complete dissolution; and then dissolving at the end the adequate quantity of nasal peptide or its pharmaceutically acceptable salt or its peptidic fragment in the solution mixture.

18. The method according to claim 17, which further comprises the step of: filtering to make the solution suitable for nasal administration and filling a mono-disposable, or multidose device system with the filtrate, more preferably with a progressive dose counting system.

19. A method for producing a pharmaceutical formulation for nasal administration, according to claim 1, in the form of reconstituted solution, said method comprising:
preparing container no. 1 with the nasal peptide either by dosing in the container the corresponding weight of powder of active nasal peptide or by preparing a suitable solution with a known concentration of the same, pouring the individually dosed volume into the container and then lyophilizing it to yield a lyophilized powder;
preparing container no. 2 comprising the solvent mixture for reconstitution, resulting from adding an adequate amount of distilled water to THAM, and optionally to methyl or/and propyl p-hydroxybenzoate, hydrochloric or citric acid and cysteine until complete dissolution;
filtering to make the solution suitable for nasal administration; and
filling container no. 2 with the filtrate.

20. The method according to claim 19, wherein container no. 1 is prepared by dosing directly in the container the corresponding weight of active nasal peptide powder or by preparing a suitable solution with a known concentration of the same, pouring the individually dosed volume directly into the container and then lyophilizing it directly in the container to yield a lyophilized powder.

21. The method according to claim 19, which further comprises the step of: preparing the reconstituted solution at the time of starting its use by pouring the solvent mixture of container no. 2 into container no. 1; mixing thoroughly by rotation until complete dissolution; and screwing the multidose device system on the neck of container no. 1, comprising the reconstituted solution.

22. The pharmaceutical formulation, according to claim 1, having a long shelf life, and when in use providing compositions of a therapeutically effective amount of active nasal peptide, its pharmaceutically acceptable salt or its peptidic fragment.

23. A method for treating a patient which comprises intranasally administering in the form of drop type or of nasal spray to said patient, a dosed volume of a formulation according to claim 1, to elicit the desired pharmacological effect.

24. The method according to claim 23, in which the administrable dose volume of the pharmaceutical formulation, comprised in a metered monodose disposable or in a multidose system thereof, is comprised between 50 microliters and 200 microliters per actuation.

25. The method according to claim 16, wherein (a) is citric acid in a concentration of 2.8 mg/ml to 6.2 mg/ml.
